(19) United States
(12) Patent Application Publication
(10) Pub. No.: US 2025/0186423 A1
(43) Pub. Date: Jun. 12, 2025

(54) METHODS OF TREATING CANCER AND OTHER CONDITIONS WITH A MU OPIOID RECEPTOR ANTAGONIST

(71) Applicant: GLYCYX MOR INC., San Francisco, CA (US)

(72) Inventors: Lorin JOHNSON, Los Altos Hills, CA (US); Jonathan MOSS, Chicago, IL (US); David TAGGART, Tucson, AZ (US)

(21) Appl. No.: 18/845,283

(22) PCT Filed: Mar. 10, 2023
(86) PCT No.: PCT/US2023/064088

§ 371 Date: Sep. 9, 2024

Related U.S. Application Data
(60) Provisional application No. 63/318,465, filed on Mar. 10, 2022.

(51) Int. Cl. A61K 31/46; A61K 31/485; A61K 39/00; A61K 45/06; A61P 35/00; A61P 43/00; C07K 16/28

(52) U.S. Cl. CPC A61K 31/46; A61K 31/485; A61K 45/06; A61P 35/00; A61P 43/00; C07K 16/2818; C07K 16/2863; A61K 2039/505

(57) ABSTRACT

Provided herein are compositions and methods of using a Mu opioid receptor (MOR) antagonist (e.g. axelopran) alone or in combination with checkpoint inhibitor (such as e.g. an inhibitor of the PD-1/PD-L1 pathway (e.g. an antibody such e.g. pem-brolizumab)) and/or an inhibitor of VEGF (e.g. bevacizumab) for reducing angiogenesis or treating cancer. Also provided herein are kits containing a MOR antagonist (e.g. axelopran) alone or in combination with checkpoint inhibitor and/or an inhibitor of VEGF for reducing angiogenesis or treating cancer.

### RELATED APPLICATIONS

This application claims benefit of U.S. Provisional Application No. 63/318,465, filed on Mar. 10, 2022, the contents of which are incorporated herein by reference in their entirety.

### TECHNICAL FIELD

This disclosure relates to using Mu Opioid Receptor (MOR) antagonists, in particular axelopran, alone or in combination with an inhibitor of the PD-1/PD-L1 pathway, and/or an inhibitor of VEGF to treat angiogenesis and cancer.

### BACKGROUND

Mu Opioid Receptor (MOR) antagonists are commonly used to treat opioid dependence and alcohol dependence. They are also used treat the side effects associated with treatment of mu opioid agonists such as e.g. morphine. Some of these side effects include induced bowel dysfunction, for example in cancer patients and in postoperative ileus.

In addition, MOR antagonists show other promising uses. For example, according to the Abstract, WO 2016061531 discloses “methods for preventing or treating tumor growth, tumor metastasis and/or abnormal proliferation of tumor cells in a subject, wherein the methods involve administration of a pharmaceutical composition comprising methylnaltrexone.” Due to their widespread use and relatively high tolerance in patients, in particular when compared to conventional chemotherapy, in view of WO 2016061531, MOR antagonists present an attractive target for treating cancer.

What is needed are additional treatments for angiogenesis and cancer.

### SUMMARY

This disclosure relates to use of Mu Opioid Receptor (MOR) antagonists, in particular axelopran, alone or in combination with an inhibitor of the PD-1/PD-L1 pathway, and/or an inhibitor of VEGF to treat angiogenesis and cancer.

One aspect of the disclosure is directed to reducing or inhibiting angiogenesis via the use of a MOR antagonist (such as e.g. axelopran). One embodiment is directed to a method of reducing or inhibiting endothelial cell growth comprising contacting an endothelial cell with a MOR antagonist. Another embodiment is directed to a method of reducing angiogenesis in a patient in need thereof comprising administering a MOR antagonist to the patient. Yet another embodiment is directed to a method of reducing angiogenesis in a cancer patient comprising administering a MOR antagonist to the cancer patient.

Another aspect of the disclosure is directed to using MOR antagonists to treat cancer or to treat specific aspects of cancer. Accordingly, one embodiment is directed to a method of reducing tumor growth in a cancer patient by administering a MOR antagonist to the cancer patient. Another embodiment is directed to a method of reducing metastasis in a cancer patient by administering a MOR antagonist to the cancer patient.

Yet another embodiment is direct to a method of improving the immune response to a tumor comprising administering a MOR antagonist to a cancer patient. In certain embodiments, the method increases infiltration of immune cells, such as e.g. NK cells, lymphocytes and/or monocytes/macrophages, into the tumor. For example, the method increases infiltration of CD3⁺, CD244⁺, and MMD⁺ immune cells.

In a specific embodiment, the disclosure is directed to a method of treating cancer by administering axelopran, naloxegol, or combinations thereof to a cancer patient. In one embodiment, the method reduces angiogenesis, tumor growth, and/or metastasis. Alternatively, the method inhibits angiogenesis, tumor growth, and/or metastasis. In some embodiments, the cancer is a melanoma, colon, pancreatic, or breast cancer.

The disclosure also provides for combination therapy using MOR antagonist in combination with a checkpoint inhibitor and/or an inhibitor of VEGF.

Yet another embodiment of the disclosure is directed to a method of treating cancer comprising administering a MOR antagonist and a checkpoint inhibitor and/or an inhibitor of VEGF to a cancer patient. In this embodiment, the MOR antagonist and the checkpoint inhibitor and/or the inhibitor of VEGF synergistically treat the cancer.

Yet another embodiment of the disclosure is directed to a method of improving the immune response to a tumor comprising administering a MOR antagonist and a checkpoint inhibitor and/or an inhibitor of VEGF to a cancer patient. In certain embodiments, the method increases infiltration of immune cells (such as NK cells, lymphocytes, and/or monocytes/macrophages) into the tumor. In a specific embodiment, the method increases infiltration of CD3⁺, CD244⁺, and MMD⁺ immune cells. In embodiments of the method, the MOR antagonist and the checkpoint inhibitor synergistically improve the immune response to the tumor.

In some embodiments, the methods and combination therapies of the disclosure are particularly useful for treating patients that are not receiving opioid treatment. In other embodiments, the methods and combination therapies of the disclosure are particularly useful for treating patients that are receiving opioid treatment.

In certain embodiments of the methods and combination therapies, the MOR antagonist comprises axelopran, naloxegol, methylnaltrexone, or combinations thereof. In specific preferred embodiments, the MOR antagonist is axelopran. In some embodiments, the MOR antagonist does not comprise (i.e. excludes) methylnaltrexone, naltrexone, and/or naloxone.

In some embodiments of the combination therapies, the checkpoint inhibitor is an inhibitor of the PD-1/PD-L1 pathway. For example, the inhibitor of the PD-1/PD-L1 pathway is an antibody, such as e.g. an antibody that binds to PD-1. In some embodiments, the antibody is a humanized antibody, such as e.g. pembrolizumab.

In other embodiments of combination therapy, the inhibitor of VEGF is an anti-VEGF antibody, such as an anti-VEGF-A antibody. In one embodiment, anti-VEGF antibody is humanized. In certain embodiments, the inhibitor of VEGF is bevacizumab. In specific embodiments of the combination therapy, the inhibitor of VEGF is intravenously injected.

In the methods or combination therapy, a variety of administration routes are used. In one embodiment, the methods or combination therapy include orally or subcutaneously administering the MOR antagonist.

The methods and combination therapy of the disclosure including administering pharmaceutical composition containing the MOR antagonist and a pharmaceutically acceptable carrier. In one embodiment, such a composition is formulated for modified release.

When used in combination therapy, the checkpoint inhibitor is administered concurrently, before, or after the MOR antagonist. Similarly, the VEGF antagonist is administered concurrently, before, or after the MOR antagonist. Furthermore, the inhibitor of VEGF is administered concurrently, before, or after the inhibitor of the PD-1/PD-L1 pathway.

Yet another embodiment of the disclosure is directed to a method of reducing tumor size comprising contacting a tumor with axelopran, naloxegol, or combinations thereof. The disclosure also relates to kits containing a MOR antagonist (e.g. axelopran) a checkpoint inhibitor and/or an inhibitor of VEGF.

Other features and advantages of the invention will be apparent from the detailed description and examples that follow.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1A and FIG. 1B show HUVEC cell tube formation in the presence of increasing MOR antagonist concentrations (0-1000 nM). Axelopran is compared to naloxegol and methylnaltrexone (MNTX).

FIG. 2A and 2B show blood vessel formation in the presence of increasing MOR antagonist (axelopran) concentrations (0, 0.1, 0.5, 1.0 mg/kg) in the chicken CAM assay in comparison to docetaxel. FIG. 2A shows a picture of CAM Angiogenesis. FIG. 2B panels A) to D) show graphs testing the impact of axelopran, bevacizumab and axelopran+bevacizumab on CAM angiogenesis.

FIG. 3A to 3C show schematic summaries of the three model systems used to assess the contribution of endogenous MOR activity on tumor progression. FIG. 3A shows a schematic summary of the melanoma model system. FIG. 3B shows a schematic summary of the breast cancer model system. FIG. 3C shows a schematic summary of the colon cancer model system.

FIG. 4A to FIG. 4C show Zebra fish larvae survival up to 72 hours in the presence of increasing axelopran concentration in the incubation water. FIG. 4A and FIG. 4B show graphs measuring the percentage survival as a function of time. FIG. 4C shows pictures of Zebra fish larvae at 24 hours, 48 hours, and 72 hours of exposure to vehicle, 100 μM axelopran, and 500 μM axelopran. Doses up the 100 μM allowed survival and development of pathological lesions at levels no different that the vehicle control.

FIG. 5 shows a schematic of time course of Zebra fish larvae development.

FIG. 6A and FIG. 6B show representative displays of melanoma tumor growth size (y-axis) (normalized as % of vehicle control) vs number of metastasis (x-axis) in zebra fish treated with 10 μM axelopran (n=18) (FIG. 6A) or vehicle control (n=32) (FIG. 6B).

FIG. 7 shows a summary of melanoma tumor progression (tumor size×#metastasis) in the presence of increasing axelopran concentrations or the checkpoint inhibitor pembrolizumab. For experiments using pembrolizumab, exogenous tumor-infiltrating lymphocytes (TILs) were also injected. The additional controls included the effect of TILs alone and with axelopran (500 nM) and pembrolizumab and TILs alone and with axelopran (500 nM). *=<0.05, **=<0.005, ***=<0.0005.

FIG. 8 shows Zebra fish larvae mortality over the 72-hour period after test article exposure at 48 hours post-fertilization.

FIG. 9 shows a schematic summary of tumor progression in the CAM assay.

FIG. 10A shows a representative two-dimensional display of breast cancer tumor growth size (normalized as % of vehicle control) vs number of metastasis (normalized as % of vehicle control) in the CAM assay treated with the following: Neg Control=vehicle (n=15)., A=1.0 mg/kg axelopran (n=13), P=pembrolizumab (2.5 mg/kg), B=bevacizumab (4 mg/kg), or the relevant combinations. Circles mark the mean of both dimensions +/−the standard deviation.

FIG. 10B shows tumor weight x metastasis for pembrolizumab, axelopran, bevacizumab, axelopran+bevacizumab, pembrolizumab+bevacizumab, and axelopran+pembrolizumab+bevacizumab.

FIG. 11 shows infiltration of chicken CD3⁺, CD244⁺ and MMD⁺ immune cells into breast cancer tumors grown on the upper CAM in the presence of axelopran, pembrolizumab, bevacizumab and combinations thereof.

FIG. 12A and FIG. 12B show MC38 colon cancer tumor growth in syngeneic mice treated with Vehicle (Control), axelopran (1 mg/kg, p.o. daily), mouse anti-PD-1 (12.5 mg/kg i.p.) or axelopran plus anti-PD-1. FIG. 12A shows tumor size (mm³) as a function of days of treatment. FIG. 12B shows the individual tumor size distribution at day 13.

FIG. 13 shows a Kaplan-Meier survival curve of mice injected with human colon cancer MC-38 cells and treated with axelopran (1 mg/kg), anti-PD-1 (2.5 mg/kg) or both agents together.

FIG. 14A and FIG. 14B show inhibition of tumor progression.

FIG. 14C shows the distribution of tumor volumes.

### DETAILED DESCRIPTION

The general description and the following detailed description are exemplary and explanatory only and are not restrictive of the invention, as defined in the appended claims. Other aspects of the present invention will be apparent to those skilled in the art in view of the detailed description of the invention as provided herein.

This disclosure is based on the discovery that MOR antagonists, in particular axelopran, can treat angiogenesis and cancer. This disclosure is also based on the surprising discovery that certain MOR antagonists (e.g. axelopran) when used in combination with an inhibitor of the PD-1/PD-L1 pathway, and/or an inhibitor of VEGF to treat angiogenesis or cancer act in synergy.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

A used herein, the term “MOR antagonist” refers to a Mu opioid receptor antagonist.

As used herein, the term “axelopran” includes axelopran which has the structure shown below:

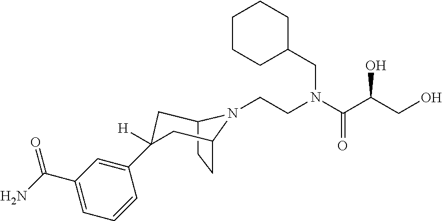 [SMILES: NC(=O)c1cccc(C2CC3CCC(C2)N3CCN(CC2CCCCC2)C(=O)[C@@H](O)CO)c1]

and which is also known as 3-[(1R,3r,5S)-8-(2-{(cyclohexylmethyl)[(2S)-2,3-dihydroxypropanoyl]amino}ethyl)-8-azabicyclo[3.2.1]octan-3-yl]benzamide or 3-[(3-endo)-8-(2-{(Cyclohexylmethyl)[(2S)-2,3-dihydroxypropanoyl]amino}ethyl)-8-azabicyclo[3.2.1]oct-3-yl]benzamide. The term axelopran also includes pharmaceutically acceptable salts of axelopran and stereoisomers of axelopran.

Furthermore, the term “axelopran” also includes derivatives of axelopran. Examples of suitable derivative of axelopran are disclosed in U.S. Pat. No. 7,622,508, the disclosure of which is incorporated as it pertains to axelopran derivatives. U.S. Pat. No. 7,622,508 discloses compounds having the core structure for Formula (I):

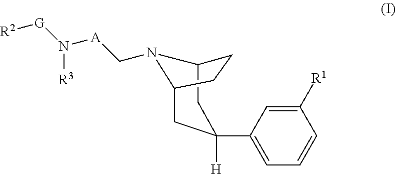

The term “salt(s) thereof” means a compound formed when a proton of an acid is replaced by a cation, such as a metal cation or an organic cation and the like. Preferably, the salt is a pharmaceutically acceptable salt. By way of example, salts of the present compounds include those wherein the compound is protonated by an inorganic or organic acid to form a cation, with the conjugate base of the inorganic or organic acid as the anionic component of the salt. Salts of interest include, but are not limited to, aluminum, ammonium, arginine, barium, benzathine, calcium, cesium, cholinate, ethylenediamine, lithium, magnesium, meglumine, procaine, N-methylglucamine, piperazine, potassium, sodium, tromethamine, zinc, N,N′-dibenzylethylene-diamine, chloroprocaine, diethanolamine, ethanolamine, piperazine, diisopropylamine, diisopropylethylamine, triethylamine and triethanolamine salts.

“Stereoisomer” and “stereoisomers” refer to compounds that have same atomic connectivity but different atomic arrangement in space. Stereoisomers include for example cis-trans isomers, E and Z isomers, enantiomers, and diastereomers. As to any of the groups disclosed herein which contain one or more substituents, it is understood, of course, that such groups do not contain any substitution or substitution patterns which are sterically impractical and/or synthetically non-feasible. All stereoisomers are intended to be included within the scope of the present disclosure.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods. A person of ordinary skill in the art can select the appropriate variation based on the context of the value.

The term “biological” or “biological sample” refers to a sample obtained from an organism or from components (e.g., cells) of an organism. The biological sample may be obtained from tumor cells or tumor tissue. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

As used herein, the terms “comprising,” “including,” “containing”, “having” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

As used herein, by “combination therapy” is meant that a first agent is administered in conjunction with another agent. “In combination with” or “In conjunction with” refers to administration of one treatment modality in addition to another treatment modality. As such, “in combination with” refers to administration of one treatment modality before, during, or after delivery of the other treatment modality to the individual. Such combinations are considered to be part of a single treatment regimen or regime. For purposes herein, a combination therapy can include a treatment regime that includes administration of a MOR antagonist and a checkpoint inhibitor each for treating the same disease or conditions, such as the same tumor or cancer. Combination therapy can include a treatment regime that includes administration of a MOR antagonist and a checkpoint inhibitor and/or a VEGF antagonist each for treating the same disease or conditions, such as the same tumor or cancer.

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term “treatment” therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

As used herein, the term “pharmaceutical composition” refers to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to intra-tumoral, intravenous, intrapleural, oral, aerosol, parenteral, ophthalmic, pulmonary, and topical administration.

The language “pharmaceutically acceptable carrier” includes a pharmaceutically acceptable salt, pharmaceutically acceptable material, composition, or carrier, such as a liquid or solid filler, diluent, excipient, solvent, or encapsulating material, involved in carrying or transporting a compound(s) (e.g. a MOR antagonist and/or a checkpoint inhibitor and/or a VEGF antagonist) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound and are physiologically acceptable to the subject. Supplementary active compounds may also be incorporated into the compositions.

As used herein, the term “effective amount” or “therapeutically effective amount” means the amount of MOR antagonist which is required to prevent the particular disease condition, or which reduces the severity of and/or ameliorates the disease condition or at least one symptom thereof or condition associated therewith.

A “subject” or “patient,” as used therein, may be a human or non-human mammal. Non-human mammals include, for example, livestock and pets, such as ovine, bovine, porcine, canine, feline, and marine mammals. Preferably, the subject is a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Therapeutic Uses of MOR Antagonists

This disclosure provides for new therapeutics uses of Mu opioid receptor (MOR) antagonists. As the examples below demonstrate, MOR antagonists are able to inhibit angiogenesis in a model for angiogenesis that is used as a model for a variety of conditions, such as those listed below. Furthermore, based on the testing, MOR antagonists alone or in combination with other therapeutic agents are able to treat cancer or symptoms related thereto.

Accordingly, one embodiment of the disclosure is directed to a method of reducing or inhibiting endothelial cell growth comprising contacting an endothelial cell with a MOR antagonist. In one embodiment, the method inhibits endothelial cell growth. In another embodiment, the method reduces endothelial cell growth.

Another embodiment of the disclosure is related to methods of reducing angiogenesis in a patient in need thereof comprising administering a MOR antagonist to the patient. In one embodiment, the methods reduce angiogenesis. In another embodiment, the methods inhibit angiogenesis. In one embodiment, the methods reduce or inhibit ocular angiogenesis. In another embodiments, the patient has psoriasis.

Another embodiment of the disclosure is related to methods of reducing angiogenesis in a patient in need thereof comprising administering a MOR antagonist to the patient suffering from suffering from an angiogenesis related disease. As used herein, an “angiogenesis related diseases” is a disease associated with abnormal angiogenesis. Examples of suitable angiogenesis related diseases include but are not limited to skin psoriasis, rheumatoid arthritis, neurodegenerative diseases, neuropathic pain, hemangiomata, collagen synthesis diseases such as Ehlers-Danlos syndrome, and portal hypertension. Further examples of angiogenesis related diseases include retinopathies (multiple pathologies, including diabetic, wet-form AMD, sickle cell retinopathy), psoriasis, rheumatoid arthritis, hemangioma, hereditary hemorrhagic teleangiectasia, Von Hippel-Lindau disease, uncontrolled vascular remodeling in ischemic diseases (including MI, others), chronic kidney disease, and Arterio-venous fistula.

Further embodiments of the disclosure are directed to methods of treating an angiogenesis-related disease comprising a MOR antagonist to a patient suffering from an angiogenesis-related disease. Example of such disease are the angiogenesis-related disease described above.

Yet another embodiment of the disclosure is directed to methods of reducing angiogenesis in a cancer patient comprising administering a MOR antagonist to the cancer patient. In one embodiment, the methods reduce angiogenesis. In another embodiment, the methods inhibit angiogenesis.

Another embodiment of the disclosure is directed to methods of reducing tumor growth in a cancer patient comprising administering a MOR antagonist to the cancer patient. The methods reduce or inhibit tumor growth.

Yet another embodiment of the disclosure is directed to methods of reducing metastasis in a cancer patient comprising administering a MOR antagonist to the cancer patient. In one embodiment, the methods inhibit metastasis. In another embodiment, the methods reduce metastasis.

An alternate embodiment of the disclosure is directed to methods of improving the immune response to a tumor comprising administering a MOR antagonist to a cancer patient. In one embodiment, the methods increase infiltration of immune cells into the tumor. In another embodiment, the methods increase infiltration of NK cells, lymphocytes and/or monocytes/macrophages. For example, the methods increase infiltration of CD3⁺, CD244⁺, and MMD⁺ immune cells, such as e.g. CD3⁺ lymphocytes, CD244⁺ natural killer cells, and MMD⁺ macrophages.

The methods of the disclosure may be used with a variety of cancers and tumors. In certain embodiments, the cancer is a melanoma, colon, or breast cancer. In other embodiments, the tumor is tumor from a patient suffering from a melanoma, pancreatic cancer, colon cancer, or breast cancer.

In preferred embodiments of the methods, the patient is not receiving opioid treatment. Accordingly, in preferred embodiments, the methods do not include (comprise) administering an opioid. In other embodiments, the patient is receiving opioid treatment.

A variety of MOR antagonists can be used in the methods of the disclosure. For example, in one embodiment, Mu receptor opioid (MOR) antagonist is axelopran, naloxegol, naldemedine, alvimopan, or combinations thereof.

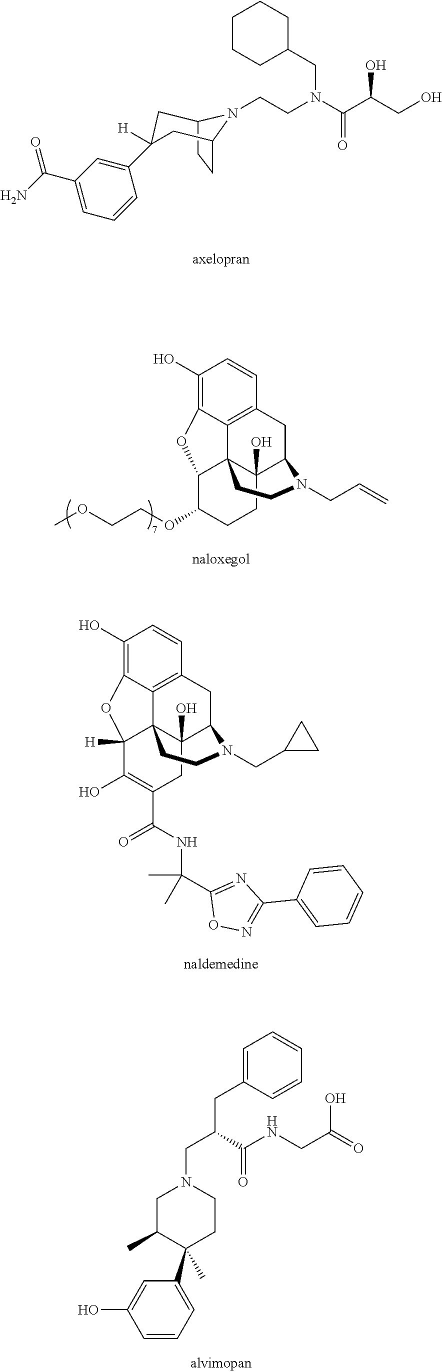

In another embodiment, the MOR antagonist is axelopran, naloxegol, or combinations thereof. In a preferred embodiment, the MOR antagonist is axelopran. In certain embodiments, the MOR antagonist is a peripheral MOR antagonist. In one embodiment, the MOR antagonist does not comprise methylnaltrexone. In another embodiment, the MOR antagonist is a peripheral MOR antagonist excluding methylnaltrexone. In another embodiment, the MOR antagonist does not comprise methylnaltrexone, naltrexone and/or naloxone.

Yet another aspect of the disclosure is directed to methods of treating cancer comprising administering axelopran, naloxegol, or combinations thereof to a cancer patient. In one embodiment, the methods include administering axelopran. In certain embodiments, the methods reduce angiogenesis, tumor growth, and/or metastasis. In other embodiments, the methods inhibit angiogenesis, tumor growth, and/or metastasis. In other embodiments, the methods improve immune response to the cancer. In alternate embodiments, the methods exclude i.e. do not comprise administering an exogenous opioid. As such, in some embodiments, the patient is not receiving opioid treatment. These methods may be used to treat a variety of cancers. In some embodiments, the cancer is a melanoma, colon, pancreatic, or breast cancer.

In certain embodiments, the methods require administering a pharmaceutical composition containing the MOR antagonist (e.g. axelopran). Examples of suitable pharmaceutical compositions are described below. In one embodiment, the pharmaceutical composition is formulated for modified release. Alternatively, the pharmaceutical composition is formulated for modified release, topical administration, or intravitreal injection. For example the pharmaceutical composition is formulated for “modified release” when it is formulated for rapid/accelerated release, site-directed release, controlled/pulsatile release, delayed release into lower gut, or sustained release.

The methods include administration of the MOR antagonist (e.g. axelopran) by a variety of routes including any of the administration routes described below. In one embodiment, the methods include orally administering the MOR antagonist. In another embodiment, the methods include subcutaneously administering the MOR antagonist. In yet another embodiment, the methods include intravitreally administering the MOR antagonist. In an alternate embodiment, the methods include topically administering the MOR antagonist.

In further embodiments, the methods including screening the patient for the presence of a cancer. These embodiments include taking a biological sample from the patient and then testing the sample for the presence of cancer cells.

Yet another embodiment of the disclosure is directed to methods of reducing tumor size comprising contacting a tumor with axelopran, naloxegol, or combinations thereof. In one embodiment, the method includes contacting the tumor with axelopran.

Other embodiments of the disclosure are directed to using a MOR antagonist (e.g. axelopran, naloxegol, or combinations thereof) in the manufacture of medicament for treating cancer. Further embodiments are directed use of a MOR antagonist (e.g. axelopran, naloxegol, or combinations thereof) for treating cancer.

### Combination Therapy

In addition, the disclosure provides for combination therapy comprising using a MOR antagonist in combination with a checkpoint inhibitor and/or a VEGF antagonist. In some embodiments, the combination therapy is used to treat angiogenesis or cancer.

Two or three drugs are administered to a subject “in combination” when the drugs are administered as part of the same course of therapy. A course of therapy refers to administration of combinations of drugs believed by the medical professional to work together additively, complementarity, synergistically, or otherwise to produce a more favorable outcome than that anticipated for administration of a single drug. A course of therapy can be for one or a few days, but more often extends for several weeks.

When two drugs are administered in combination, a variety of schedules can be used. In one case, for example and without limitation, Drug 1 (e.g. the MOR antagonist) is first administered prior to administration of Drug 2 (e.g. the checkpoint inhibitor and/or the VEGF antagonist), and treatment with Drug 1 is continued throughout the course of administration of Drug 2; alternatively Drug 1 is administered after the initiation or completion of Drug 2 therapy; alternatively, Drug 1 is first administered contemporaneously with the initiation of the other cancer therapy. As used in this context, “contemporaneously” means the two drugs are administered the same day, or on consecutive days.

Although in principle certain drugs can be co-formulated, in general they are administered in separate compositions. Similarly, although certain drugs can be administered simultaneously, more often (especially for drugs administered by infusion) drugs are administered at different times on the same day, on consecutive days, or according to another schedule.

Accordingly, one embodiment of the disclosure is directed to methods of treating cancer comprising administering a MOR antagonist and a checkpoint inhibitor and/or an inhibitor of VEGF (e.g. a MOR antagonist in combination with a checkpoint inhibitor and/or an inhibitor of VEGF (e.g. bevacizumab)) to a cancer patient. In preferred embodiments of these methods, the MOR antagonist and the checkpoint inhibitor and/or an inhibitor of VEGF synergistically treat the cancer. In one embodiment, the MOR antagonist and the checkpoint inhibitor (an anti-PD1 antibody) synergistically treat cancer. One specific embodiment is a method of treating cancer with a synergistic combination of axelopran and pembrolizumab (an anti-PD1 antibody). Another specific embodiment is a method of treating cancer with a synergistic combination of axelopran, bevacizumab (an anti-VEGF antibody), and pembrolizumab (an anti-PD1 antibody). In other embodiments, the methods include administering the MOR antagonist in combination with the checkpoint inhibitor. In alternate embodiments, the methods include administering the MOR antagonist in combination with the VEGF antagonist.

Another embodiment of the disclosure is directed methods improving the immune response to a tumor comprising administering a MOR antagonist and a checkpoint inhibitor and/or an inhibitor of VEGF to a cancer patient. In some embodiments, the methods comprise administering a MOR antagonist in combination with a checkpoint inhibitor. In other embodiments, the methods comprise administering a MOR antagonist in combination with a VEGF antagonist. In certain embodiments, the methods increase infiltration of immune cells into the tumor. In specific embodiments, the methods increase infiltration of NK cells, lymphocytes and/or monocytes/macrophages. Alternatively, the methods increase infiltration of CD3⁺, CD244⁺, and MMD⁺ immune cells. In certain embodiments, the MOR antagonist and the checkpoint inhibitor synergistically improve the immune response to the tumor.

A variety of MOR antagonists may be used. In some embodiments, the MOR antagonist comprises axelopran, naloxegol, methylnaltrexone, or combinations thereof. In other embodiments, the MOR antagonist is axelopran.

In certain specific embodiments of the combination therapy, the MOR antagonist does not comprise methylnaltrexone and/or naloxone.

A variety of checkpoint inhibitors can be used in combination with the MOR antagonists such as e.g. axelopran. In certain embodiments, the checkpoint inhibitor is an inhibitor of the PD-1/PD-L1 pathway such as e.g. an antibody. In other embodiments, the checkpoint inhibitor is an inhibitor of: CTLA-4; LAG-3; TIM-3; B7-H3; B7-H4; A2aR; NKG21; PVRIG; PVRL2; CEACAM 1; CEACAM 5; CEACAM 6; FAK; CCL2/CCR2: LIF: CD45: CSF-1: SEMA4D: CLEVER-1: OX-40: IL-1; IL-6; or IL-8. In certain embodiments, the checkpoint inhibitor is an antibody. Other examples of suitable checkpoint inhibitors are disclosed in Marin-Acevedo et al. J Hematol Oncol (2021) 14:45, the disclosure of which is incorporated as it pertains to checkpoint inhibitors. In some embodiments, the checkpoint inhibitor is an antibody that binds to PD-1 such as e.g. a humanized antibody. In one embodiment, the checkpoint inhibitor is pembrolizumab.

In some embodiments of the combination therapy, the methods include orally or subcutaneously administering the MOR antagonist. In other embodiments, the method comprises administering a pharmaceutical composition comprising the MOR antagonist and a pharmaceutically acceptable carrier as described herein. For example, the method includes administering a pharmaceutical composition formulated for modified release. For example, the pharmaceutical composition is formulated for “modified release” when it is formulated for rapid/accelerated release, site-directed release, controlled/pulsatile release, delayed release into lower gut, or sustained release. When used, the checkpoint inhibitor is administered concurrently, before, or after the MOR antagonist (e.g. axelopran).

In certain embodiments, the methods include administering an inhibitor of angiogenesis. The inhibitor of angiogenesis can be used alone or in combination with a checkpoint inhibitor. In some embodiments, the inhibitor of angiogenesis is an inhibitor of VEGF. The VEGF inhibitor can be used alone or in combination with the checkpoint inhibitor (e.g. an antibody that binds to PD-1 such as e.g. pembrolizumab). In some embodiments, the VEGF inhibitor is an anti-VEGF antibody, such as e.g. an anti-VEGF-A antibody. In some embodiments, the VEGF inhibitor is a humanized antibody. the antibody is an anti-VEGF-A antibody. In other embodiments, the inhibitor of VEGF is bevacizumab. Other suitable inhibitors of VEGF include but are not limited to Ziv-aflibercept, aflibercept, ranibizumab, pegaptanib, or faricimab. In some embodiments of the methods (combination therapy), the inhibitor of VEGF is intravenously injected. When used, the inhibitor of VEGF is administered concurrently, before, or after the MOR antagonist. In other embodiments, when used in combination with a checkpoint inhibitor, the inhibitor of VEGF is administered concurrently, before, or after the check point inhibitor such as e.g. the inhibitor of the PD- 1/PD-L1 pathway.

In some embodiments, the disclosure is directed to use of a MOR antagonist, as described above, and an inhibitor of the PD-1/PD-L1 pathway, as described above, in the manufacture of a medicament for treating cancer. The disclosure is also directed use of a MOR antagonist, as described above, and an inhibitor of the PD-1/PD-L1 pathway, as described above, in the manufacture of a kit for treating cancer. Additionally, the disclosure is directed to use of a MOR antagonist, as described above, and an inhibitor of the PD-1/PD-L1 pathway, as described above, for treating cancer.

In further embodiments, the disclosure is directed to use of a MOR antagonist, as described above, and an inhibitor of VEGF, as described above, in the manufacture of a medicament for treating cancer. Alternatively, the disclosure is directed to use of a MOR antagonist, as described above, and an inhibitor of VEGF, as described above, for treating cancer. Additionally, the disclosure is directed to a MOR antagonist, as described above, and an inhibitor of VEGF, as described above, in the manufacture of a medicament for treating cancer. Furthermore, the disclosure is directed to use of a MOR antagonist, as described above, and an inhibitor of VEGF, as described above, in the manufacture of a kit for treating cancer.

Additionally, the disclosure is directed to use of a MOR antagonist, as described above, an inhibitor of the PD-1/PD-L1 pathway, as described above, and an inhibitor of VEGF, as described above, in the manufacture of a medicament for treating cancer. Furthermore, the disclosure is directed to use of a MOR antagonist, as described above, an inhibitor of the PD-1/PD-L1 pathway, as described above, and an inhibitor of VEGF, as described above, for treating cancer.

In addition, in one embodiment, the disclosure is directed to a combination comprising a MOR antagonist, as described above (e.g. axelopran), and an inhibitor of the PD-1/PD-L1 pathway (e.g. pembrolizumab), as described above, for use in a method of treating cancer, the method comprising administering the combination to a subject in need thereof. In certain embodiments, the combination also includes an inhibitor of VEGF, as described above (e.g. bevacizumab).

In specific embodiments of the methods and uses described above, the MOR antagonist is axelopran and the checkpoint inhibitor is an anti-PD1 antibody, such, as for example, pembrolizumab. In alternate embodiments of the methods and uses described above, axelopran and pembrolizumab act in synergy. In other embodiments of the methods and uses described above, the MOR antagonist is axelopran, the checkpoint inhibitor is an anti-PD1 antibody, such, as for example, pembrolizumab, and the inhibitor of VEGF is bevacizumab. In further embodiments of the methods and uses described above, axelopran, pembrolizumab, and bevacizumab act in synergy.

### Compositions and Pharmaceutical Compositions

One embodiment is a composition comprising a synergistic amount of a MOR antagonist and an inhibitor of the PD-1/PD-L1 pathway. In certain embodiments of the composition, the MOR antagonist comprises axelopran, naloxegol, methylnaltrexone, or combinations thereof. In one embodiment, the MOR antagonist is axelopran. The inhibitor of the PD-1/PD-L1 pathway is an antibody, such as an antibody that binds to PD-1 (e.g. pembrolizumab). The antibody can be humanized.

In some embodiments, the invention is directed to pharmaceutical compositions containing a MOR antagonist configured for use in the methods described herein. In one preferred embodiment, the MOR antagonist is axelopran.

In certain embodiments, the invention is directed to pharmaceutical compositions comprising a MOR antagonist and a checkpoint inhibitor and/or a VEGF antagonist. In another embodiment, the invention is directed a pharmaceutical composition comprising a MOR antagonist and a separate pharmaceutical composition comprising a checkpoint inhibitor and/or a VEGF antagonist.

Provided herein is a pharmaceutical composition for treating a cancer in a subject in need thereof. The pharmaceutical composition comprises a MOR antagonist, and a pharmaceutically acceptable carrier. In certain embodiments, the pharmaceutical composition comprises a checkpoint inhibitor and/or a VEGF antagonist.

In one embodiment, the pharmaceutical composition comprises axelopran, pembrolizumab, and/or bevacizumab. In another embodiment, the pharmaceutical composition comprises axelopran and pembrolizumab.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single-or multi-dose unit.

The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs. In one embodiment, the subject is a human or a non-human mammal such as but not limited to an equine, an ovine, a bovine, a porcine, a canine, a feline and a murine. In one embodiment, the subject is a human.

In one embodiment, the compositions are formulated using one or more pharmaceutically acceptable excipients or carriers. In one aspect a pharmaceutical composition is disclosed for treating a cancer in a subject. The pharmaceutical composition comprises a MOR antagonist. In another embodiment, the pharmaceutical compositions also contain a checkpoint inhibitor, a VEGF antagonist, or a combination thereof. Pharmaceutically acceptable carriers, which are useful, include, but are not limited to, glycerol, water, saline, ethanol, and other pharmaceutically acceptable salt solutions such as phosphates and salts of organic acids. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

The compositions may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea, and combinations thereof. A particularly preferred preservative is a combination of about 0.5% to 2.0% benzyl alcohol and 0.05% to 0.5% sorbic acid.

The compositions may include an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

The pharmaceutical composition disclosed herein may be used in combination with an additional therapeutic agent such as an anti-tumor agent, including but not limited to a chemotherapeutic agent, an anti-cell proliferation agent or any combination thereof. For example, any conventional chemotherapeutic agents of the following non- limiting exemplary classes are included in the invention: alkylating agents; nitrosoureas; antimetabolites; antitumor antibiotics; plant alkyloids; taxanes; hormonal agents; and miscellaneous agents. In another aspect, the pharmaceutical composition disclosed herein may be used in combination with a radiation therapy.

### Administration/Dosing

In certain embodiments of the invention, the MOR antagonist, and the checkpoint inhibitor and/or the VEGF antagonist are administered at the same time. In other embodiments, the checkpoint inhibitor and/or the VEGF antagonist is administered before the MOR antagonist is administered. In another embodiment, the checkpoint inhibitor and/or the VEGF antagonist is administered after MOR antagonist administration.

The regimen of administration may affect what constitutes an effective amount. For example, the therapeutic formulations may be administered to the patient subject either prior to or after a surgical intervention related to cancer, or shortly after the patient was diagnosed with cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

The volume of the composition can be any volume, and can be for single or multiple dosage administration, including, but not limited to, from or from about 0.01 mL to 100 mL, 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL, or 0.5 mL to 5 mL, each inclusive.

Administration of the compositions of the present invention to a patient subject, preferably a mammal, more preferably a human, may be carried out using known procedures, at dosages and for periods of time effective to treat cancer in the subject. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily, or the dose may be proportionally reduced as indicated by the exigencies of the therapeutic situation. A non-limiting example of an effective dose range for a therapeutic compound is from about 0.01 to about 50 mg/kg of body weight/per day.

The MOR antagonist and the checkpoint inhibitor and/or the VEGF receptor antagonist can be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non- limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal. Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient. A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for treating cancer or other conditions in a patient.

### Routes of Administration

One skilled in the art will recognize that although more than one route can be used for administration, a particular route can provide a more immediate and more effective reaction than another route. In certain embodiments of the invention, the MOR antagonist, and the checkpoint inhibitor and/or the VEGF antagonist are administered via the same route of administration. In other embodiments, the MOR antagonist, and the checkpoint inhibitor and/or the VEGF antagonist are administered via different routes of administration.

Routes of administration of the disclosed compositions (containing a MOR antagonist or MOR antagonist, and the checkpoint inhibitor and/or the VEGF antagonist) include inhalational, oral, nasal, rectal, parenteral, sublingual, transdermal, transmucosal (e.g., sublingual, lingual, (trans) buccal, (trans) urethral, vaginal (e.g., trans-and perivaginally), (intra) nasal, and (trans) rectal), intravesical, intrapulmonary, intraduodenal, intragastrical, intrathecal, subcutaneous, intramuscular, intradermal, intra-arterial, intravenous, intrabronchial, inhalation, and topical administration. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder, or aerosolized formulations for inhalation, compositions, and formulations for intravesical administration and the like. It should be understood that the formulations and compositions that would be useful in the present invention are not limited to the particular formulations and compositions that are described herein. In one embodiment, the MOR antagonist treatment and/or treatment with the checkpoint inhibitor and/or the VEGF antagonist comprises an administration route selected from the group consisting of inhalation, oral, rectal, vaginal, parenteral, topical, transdermal, pulmonary, intranasal, buccal, ophthalmic, intra-hepatic arterial, intrapleural, intrathecal, intra-tumoral, intravenal, and any combination thereof.

### Kits

The invention also includes kits containing the MOR antagonist, checkpoint inhibitor, and/or VEGF antagonist whereby the kits are used to treat a cancer. In one embodiment, the kit comprises a pharmaceutical a pharmaceutical composition comprising the MOR antagonist and a pharmaceutically acceptable carrier. In one embodiment, the kit includes a pharmaceutical composition comprising a Mu receptor (e.g. axelopran) antagonist and the checkpoint inhibitor (e.g. pembrolizumab). In certain embodiments, the pharmaceutical composition also contains an inhibitor of VEGF. The pharmaceutical composition can be formulated for modified release, topical administration, or intravitreal injection.

In one embodiment, the kit includes a pharmaceutical composition comprising a Mu receptor (e.g. axelopran) antagonist and the checkpoint inhibitor (e.g. pembrolizumab). The kit also includes a separate pharmaceutical composition comprising the VEGF inhibitor (e.g. pembrolizumab).

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention and are not to be construed as limiting in any way the remainder of the disclosure.

### Example 1

### Effect of MOR Antagonists on Angiogenesis

Two independent model systems were used to explore the role of MOR and endogenous opioids on angiogenesis. Human umbilical vein endothelial cells (HUVEC) in culture initiate angiogenesis through tube formation when cultured on an appropriate extracellular matrix. In a living system, the chicken egg chorioallantoic membrane assay (CAM) allows direct visualization of new blood vessel formation.

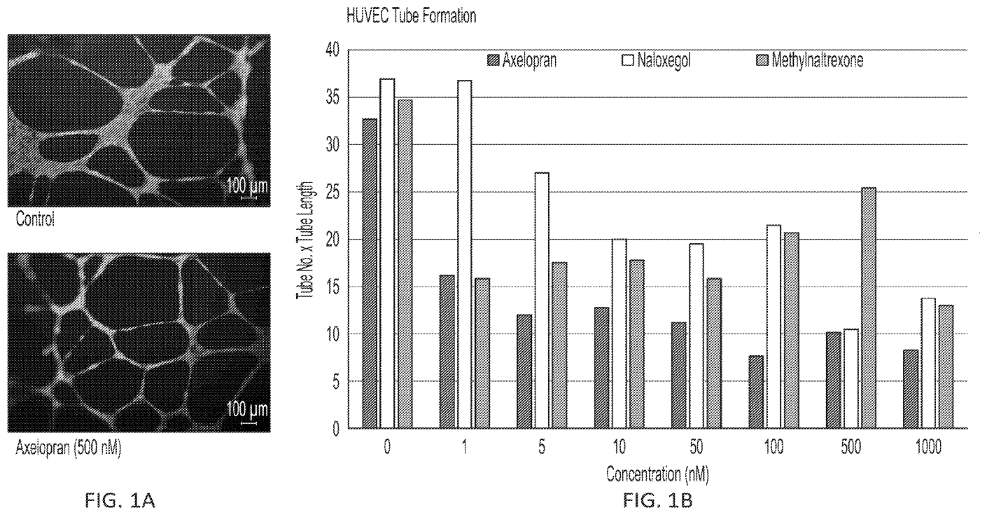

Specifically, the HUVEC cell tube formation was assessed in the presence of various doses (0-1000 nM) of three different Mu opioid receptor antagonists (axelopran, naloxegol, and methylnaltrexone (MNTX)). For this testing, the HUVEC cells were exposed to the various MOR antagonist concentrations and incubated under the following conditions: Briefly, 50 μL of Matrigel in 96 well plates was loaded to make one layer of extracellular matrix. The plate was kept for 1 hour at 37° C. HUVEC cells were seeded on top of Matrigel (0.02×10⁶ cells/well) and waited for 4 h to form tube formation. After four hours, plates were observed for tube formation. Once tubes formed, cells were treated with increasing amounts of the test compounds (axelopran, naloxegol, and MNTX) and incubated for 18 hours. Cells were stained with Calcein AM and images were captured. The data analysis was performed using NIH imageJ software. Tube length was measure and data was plotted using prism software. The results of the testing are shown in FIG. 1A and FIG. 1B.

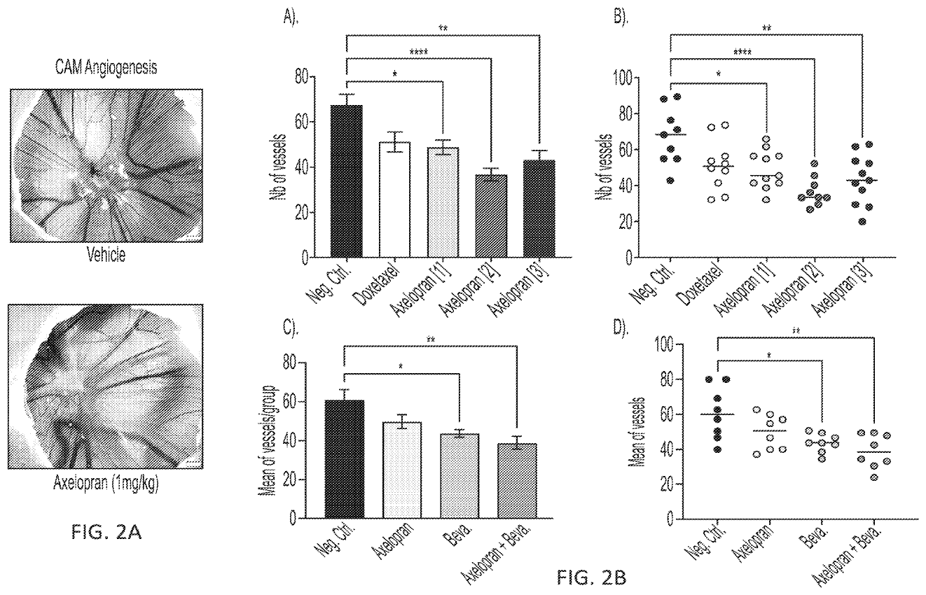

Furthermore, a chicken egg chorioallantoic membrane assay was conducted to determine blood vessel formation in the presence of increasing MOR antagonist (axelopran) concentrations (0, 0.1, 0.5, 1.0 mg/kg). Docetaxel was used as a control. For the testing CAM membranes were incubated in the presence of the MOR antagonist or docetaxel under the following conditions: Fertilized White Leghorn eggs were incubated at 37.5° C. with 50% relative humidity for 9 days. At that moment (E9), the CAM was dropped down by drilling a small hole through the eggshell into the air sac, and a 1 cm window was cut in the eggshell above the CAM. At least 20 eggs (depending on embryo surviving rate after 9 days of development, there could be more than 20 eggs per group) were grafted for each group. MDA-MB-231 tumor cell line was cultivated in D-MEM medium supplemented with 10% FBS and 1% penicillin/streptomycin. On day E9, cells were detached with trypsin, washed with complete medium and suspended in graft medium. An inoculum of 1 ×10⁶ cells was added onto the CAM of each egg (E9) and then eggs were randomized into groups. On day E16, a picture of the upper CAM (with tumor) was taken (n=8 per group). The number of blood vessels that reach the tumor and feed the tumor proliferation were counted in triplicate to evaluate tumor angiogenesis. Tumor vessel counting was performed for 8 eggs per group. The results of this testing are shown in FIG. 2A and FIG. 2B.

Using both of these assays, the testing in this Example shows that axelopran, naloxegol, and methylnaltrexone inhibit angiogenesis in the absence of exogenous opioids. In contrast, Feng et al. observed that “[o]ur findings that [the MOR antagonist] naloxone neither antagonizes opioids-induced angiogenesis nor induces angiogenesis by itself are also observed in human microvascular endothelial cells exposed to morphine and naloxone.” Feng et al. BMC Anesthesiol (2021)21:257 at 9-10.

According to Goel et al., “[g]iven that hypoxia, inflammation, and dysregulation of the extracellular matrix can all lead to aberrant vessel morphology and function, it is not surprising that tortuosity and ‘abnormalization’ of the vasculature have also been reported in a number of other nonmalignant diseases. These include skin psoriasis, rheumatoid arthritis, neurodegenerative diseases and neuropathic pain model, hemangiomata, collagen synthesis diseases such as Ehlers-Danlos syndrome, and portal hypertension.” Goel et al., Physiol Rev. 2011 July; 91(3): 1071-1121. Accordingly, the testing in this Example establishes the MOR antagonists are able to treat a variety of diseases which involve angiogenesis.

### Example 2

### Effect of MOR Antagonists on Tumor Progression

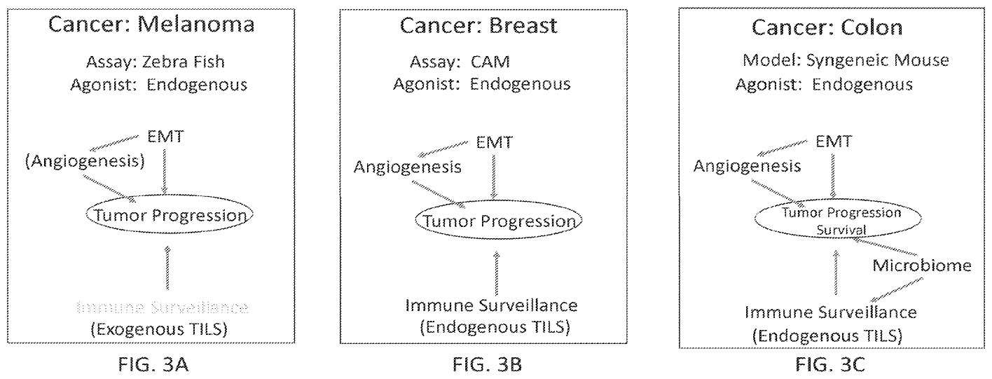

Three unique model systems were chosen to study the relevance of endogenous MOR activity on tumor progression. Each system allows the study of different aspects of the host-tumor interaction (FIG. 3A-3C).

Zebra fish larvae (FIG. 3A) allow direct visualization of injected tumor cell growth and metastasis (collectively tumor progression) using fluorescently label tumor cells. At this developmental stage both the zebra fish immune system and gut microbiome are immature or lacking. Co-injection of exogenous tumor infiltrating lymphocytes (ex-TILS) allows the separate study of immune surveillance in the absence of a microbiome.

The chicken egg chorioallantoic membrane assay (FIG. 3B) allows for the study of tumor progression of injected tumor cells in the presence of active endogenous immune surveillance, but again in the absence of an active gut microbiome.

The syngeneic mouse model assay (FIG. 3B) allows a complete system where tumor progression is quantified in the presence of endogenous immune surveillance and an active gut microbiome.

### Zebra Fish Larvae

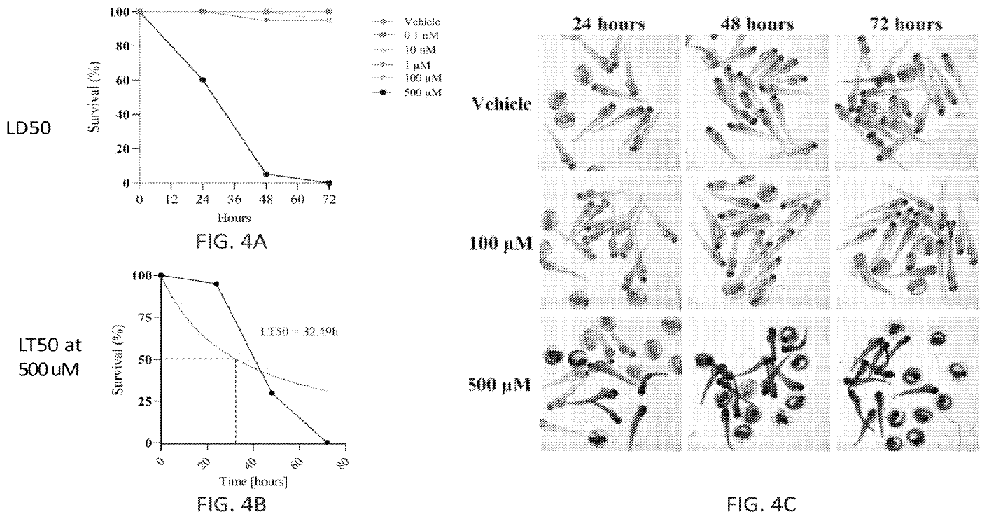

The MOR antagonist axelopran was first tested for toxicity of zebra fish development to ensure that the doses were studied in a range that did not interfere with the normal fish ontogeny. Transgenic Tg(fli1: EGFP)y1 zebrafish embryos were used for these studies. The results of this testing are shown in FIG. 4A and FIG. 4B, which show Zebra fish larvae survival up to 72 hours in the presence of increasing axelopran concentration in the incubation water. FIG. 4A and FIG. 4B shows graphs measuring the percentage survival as a function of time. FIG. 4C shows pictures of Zebra fish larvae at 24 hours, 48 hours, and 72 hours of exposure to vehicle, 100 μM axelopran, and 500 μM axelopran. Doses up the 100 μM allowed survival and development of pathological lesions at levels no different that the vehicle control.

Accordingly, the toxicity screen testing demonstrates that doses up to 100 μM of axelopran in the incubation water of the larvae did not result in larvae death and showed limited abnormal pathology. Based on the toxicity screening, for tumor progression analysis, doses up to 10 μM were utilized.

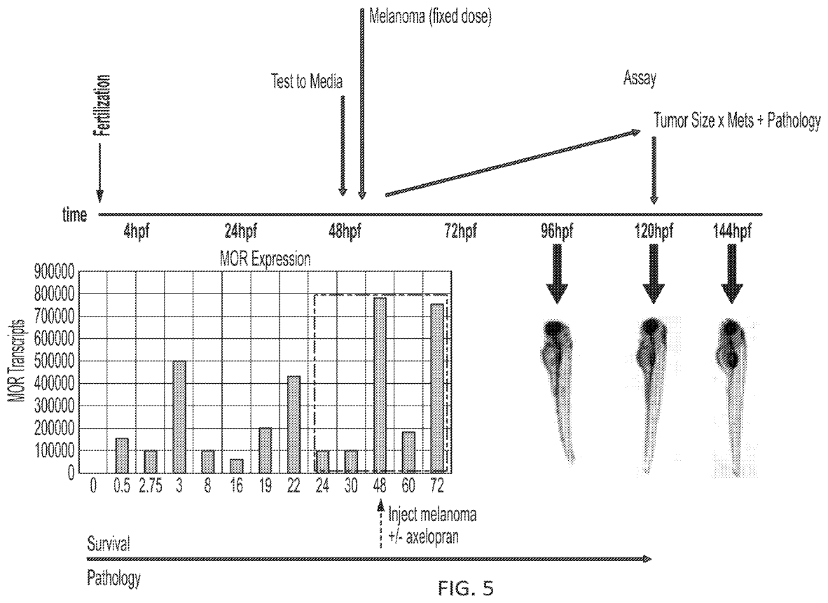

Having established suitable doses for studying axelopran, a tumor progression analysis was conducted. The general protocol for the tumor progression analysis is shown in FIG. 5. Specifically, FIG. 5 shows a schematic of time course of Zebra fish larvae development.

The toxicity screening period covered the first 72 hours of development. The efficacy evaluation of Axelopran was made using M-001 melanoma cells and matching TILs. Two-day old zebrafish embryos were subcutaneously injected into the perivitelline space with approximately 700 M-001 cells labelled with Dil red fluorescent dye and intravenously injected with TILs with or without pembrolizumab antibody. At the same time, MOR-antagonist test doses were added to the water. Following an additional 72 hours of further development, the fish were assayed for tumor growth (size of original tumor implant) and metastasis disseminated to the distal caudal venous plexus (CVP) three days after implantation.

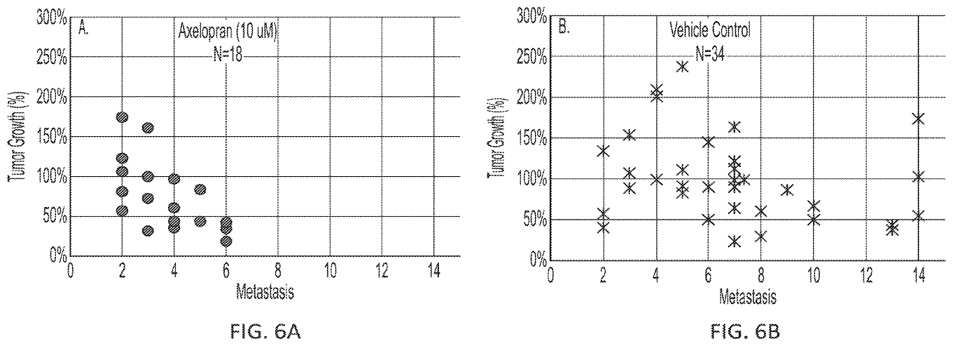

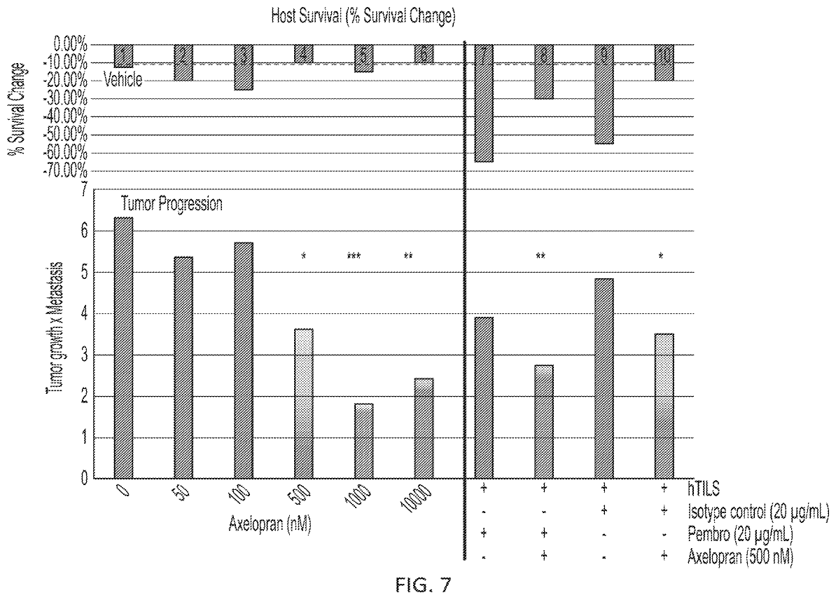

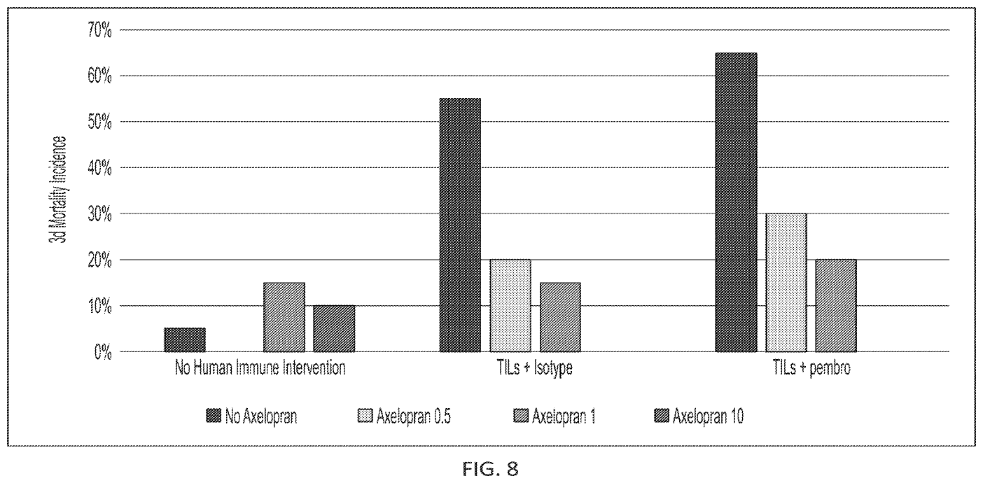

The results of this testing are shown in FIG. 6A, FIG. 6B, FIG. 7, and FIG. 8 as well as in Table 2-1. FIG. 6A and FIG. 6B show representative displays of melanoma tumor growth size (y-axis) (normalized as % of vehicle control) vs number of metastasis (x-axis) in zebra fish treated with 10 μM axelopran (n=18) (FIG. 6A) or vehicle control (n=32) (FIG. 6B). As is evident from FIG. 6A and FIG. 6B, exposure to 10 μM axelopran significantly reduced metastasis, indicating that in this model, axelopran is effective to treat cancer.

The testing described above was repeated studying the effects of various doses of axelopran (0, 50 nM, 100 nM, 500 nM, and 1000 nM) alone, pembrolizumab alone, or pembrolizumab in combination with axelopran. The results of this testing are shown in FIG. 7, which depicts a summary of melanoma tumor progression (tumor size×#metastasis) in the presence of increasing axelopran concentrations or the checkpoint inhibitor pembrolizumab. For experiments using pembrolizumab, exogenous tumor-infiltrating lymphocytes (TILs) were also injected. The additional controls included the effect of TILs alone and with axelopran (500 nM) and pembrolizumab and TILs alone and with axelopran (500 nM). *=<0.05, **=<0.005, ***=<0.0005. The testing is also summarized in Table 2-1 below.

TABLE 2-1
Melanoma tumor progression index (tumor size × metastasis)
in Zebra Fish larvae over 72 hours of growth. Axelopran
dose response and combination with pembrolizumab (Pembro)

| | | | | | | Pembro | Pembro | | |
|---|---|---|---|---|---|---|---|---|---|
| | | Axelopran (nM) | | | | +TILS | +TILS | +TILS | +TILS |
| | Vehicle | 100 | 500 | 1000 | 10000 | — | 500 nM | — | 500 nM |
| Mean | 6.34 | 5.71 | 3.62 | 1.85 | 2.41 | 3.89 | 2.73 | 4.83 | 3.52 |
| StDev | 4.36 | 6.63 | 5.00 | 1.77 | 1.09 | 3.58 | 2.14 | 7.66 | 2.71 |
| N | 35 | 15 | 36 | 17 | 18 | 7 | 14 | 9 | 16 |
| P-Value | | n.s. | 0.033 | 0.0004 | 0.0011 | n.s. | 0.0098 | n.s. | 0.0394 |

For study investigating combination therapy, the Zebra fish larvae mortality was also investigated. While no significant mortality was seen with axelopran alone, mortality in the presence of exogenous TILs and TILs plus pembrolizumab was significantly increased (to 63%) while axelopran significantly reduced this mortality when added with TILS and TILS plus pembrolizumab (FIG. 8).

### Chicken Egg—Chorioallantoic Membrane (CAM) Assay

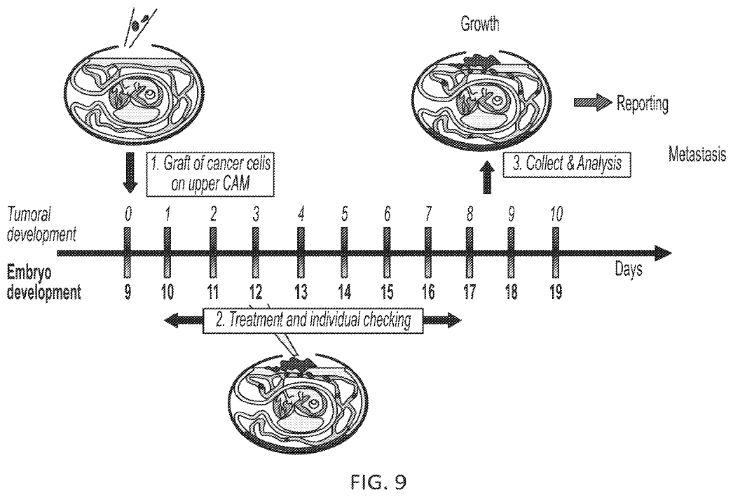

Breast cancer cell tumor progression was assessed in the chicken egg, chorioallantoic membrane assay. The experimental timing is shown below in FIG. 9. Fertilized chicken eggs were incubated for 9 days. The MDA-MB-231 tumor cell line was cultivated in D-MEM medium supplemented with 10% FBS and 1% penicillin/streptomycin. On day E9, cells were detached with trypsin, washed with complete medium and suspended in graft medium. An inoculum of 1×10⁶ cells was added onto the CAM of each egg (E9) and then eggs were randomized into groups. Tumor cells (breast cancer MDA-MB231 cells (10⁶)) were then seeded on the upper CAM and further incubated for 3 three days. Test agents were then added to the same site at selected concentrations. Following five days of further incubation the growing tumor was excised, freed of CAM tissue, and weighed. A comparable region of the lower CAM was harvested and screened for human Alu sequences by quantitative PCR to measure a relative metastasis value for human cells migrating from the upper CAM to the lower CAM.

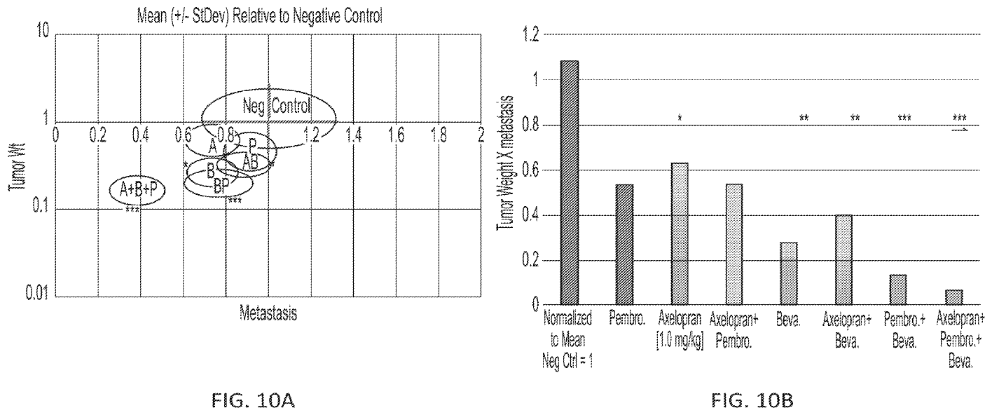

For this testing, the chicken eggs were treated with: axelopran; pembrolizumab (Pembro); axelopran+pembrolizumab; bevacizumab (Beva); axelopran+bevacizumab; pembrolizumab+bevacizumab; and axelopran+pembrolizumab+bevacizumab. Axelopran was used at dose of 1.0 mg/kg; pembrolizumab was used at a dose of 2.5 mg/kg; and bevacizumab was used at a dose of 4 mg/kg. The results of this testing are shown in FIG. 10A, 10B, 11 and Table 2-2.

Representative two-dimensional displays of breast cancer tumor growth size (normalized as % of vehicle control) vs number of metastasis (normalized as % of vehicle control) in the CAM assay treated with the following: Neg Control=vehicle (n=15), A=1.0 mg/kg axelopran (n=13), P=pembrolizumab (2.5 mg/kg), B=bevacizumab (4 mg/kg), or the relevant combinations are shown in FIG. 10A. Circles mark the mean of both dimensions±the standard deviation.

Tumor weight x metastasis for pembrolizumab, axelopran, bevacizumab, axelopran+bevacizumab, pembrolizumab+bevacizumab, and axelopran+pembrolizumab+bevacizumab are shown in FIG. 10B.

TABLE 2-2
Breast cancer tumor progression index (tumor size × metastasis) in the CAM model over 5 days of growth
in the upper CAM. Data are normalized as fraction of negative control for both tumor size and metastasis.

| | | | | | | | | Axelopran + |
|---|---|---|---|---|---|---|---|---|
| Gr. | | | Axelopran | Axelopran + | | Axelopran + | Pembro + | Pembro + |
| Description | Neg. Ctrl. | Pembro | 1 mg/kg | Pembro | Beva | Beva | Beva | Beva |
| Mean | 1.08 | 0.54 | 0.63 | 0.54 | 0.28 | 0.40 | 0.13 | 0.06 |
| SD | 0.76 | 0.31 | 0.32 | 0.34 | 0.09 | 0.16 | 0.08 | 0.09 |
| N | 16 | 8 | 16 | 7 | 7 | 6 | 8 | 8 |
| P-value | vs Neg | 0.071 | 0.039 | 0.090 | 0.012 | 0.046 | 0.002 | 0.001 |
| | Control | | | | | | | |

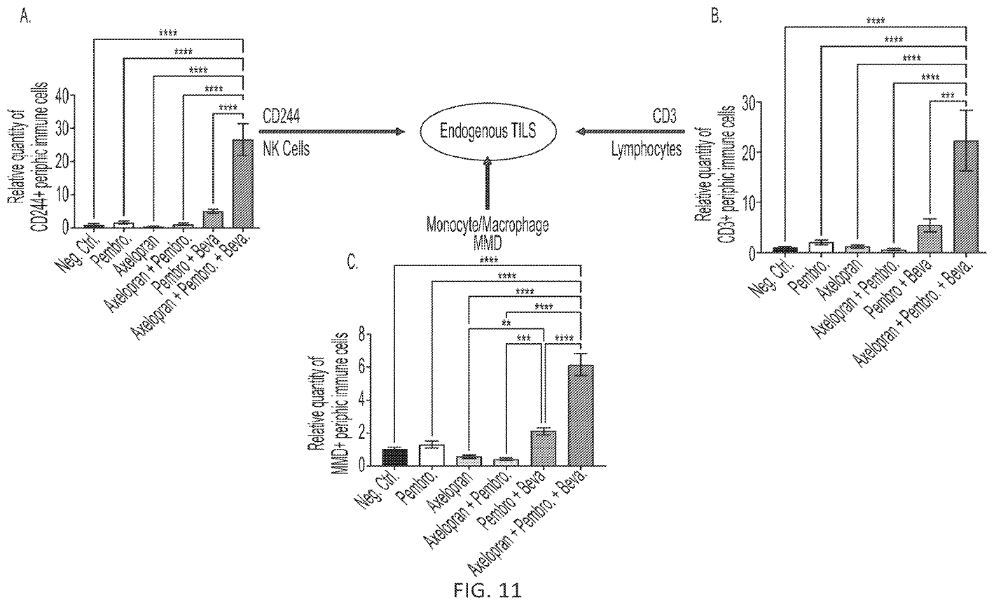

Infiltration of host immune cells into the tumor environment is required for host reduction of tumor progression. Among the tumors collected at E18, after tumor weighing, 10 tumors per group (n=10) were used for RNA extraction, in 6 groups (Gr. 1, 2, 3, 4, 7, and 8). After that, extracted RNA was analyzed by RT-qPCR with specific primers for chicken CD3, MMD and CD244 sequences. For all points done in qPCR, expression of human GAPDH was also analyzed, as reference gene expression, and used to normalize immune biomarker expression between samples. Calculation of Cq for each sample, mean Cq and relative amounts of immune cells for each group were directly managed by the Bio- Rad CFX Maestro software (see FIG. 11).

The infiltration of chicken CD3⁺, CD244⁺, and MMD⁺ immune cells into breast cancer tumors grown on the upper CAM in the presence of axelopran, pembrolizumab, bevacizumab and combinations are shown in FIG. 11. In all cases, axelopran synergistically enhanced the infiltration of CD3⁺ lymphocytes, CD244⁺ natural killer cells and MMD⁺ macrophages into the tumor tissue when used in the presence of bevacizumab and pembrolizumab.

### Syngeneic Mouse Plus Colon Cancer

Having studied the effect of axelopran alone or in combination with an anti- PD1 antagonist (Pembro) in a model without a gut microbiome, further studies on the impact of axelopran alone or in combination with an anti-PD1 antagonist in a model with a microbiome were conducted.

Mouse colon cancer (MC38) cells were amplified in vitro prior to implantation. On the day of injection, cells were harvested, counted including a trypan blue viability dye (cut-off 80%), and resuspended in PBS at the appropriate concentration. The cells were injected subcutaneously in the right flank of Female mice (C57BL6), 5 weeks of age (18-22 gm body weight) at 5.106 cells/mouse in 200 μL PBS within 30 minutes after harvesting. After 15-18 days of tumor growth, mice were randomized into treatment groups at 10 animals/group. Animals were weighed thrice weekly and tumor growth measured using callipers. The tumour volume (TV) was extrapolated to a sphere using the formula shown below:

$$T V = \frac{4}{3} π {r}^{3}$$

by calculating the mean radius from the two measurements.

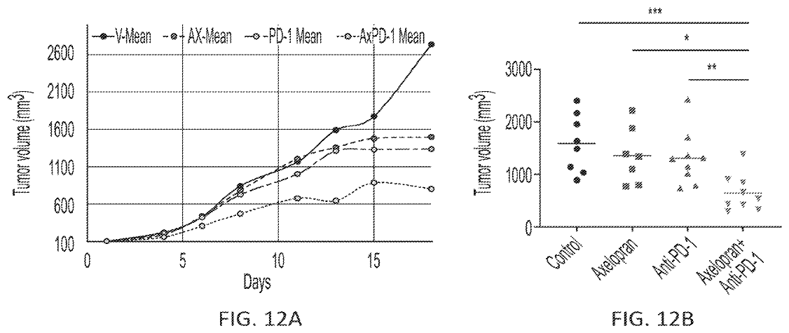

The results of these studies are shown in FIG. 12A, 12B, 13 as well as Table 2-3. The tumor growth curves in animals treated with the MOR antagonist, axelopran, mouse anti-PD-1 or a combination of both treatments are shown in FIGS. 12A and 12B. FIG. 12A and FIG. 12B show MC38 colon cancer tumor growth in syngeneic mice treated with Vehicle (Control), axelopran (1 mg/kg, p.o. daily), mouse anti-PD-1 (12.5 mg/kg i.p.,3×-weekly) or axelopran plus anti-PD-1. FIG. 12A shows tumor size (mm³) as a function of days of treatment. FIG. 12B shows the individual tumor size distribution at day 13.

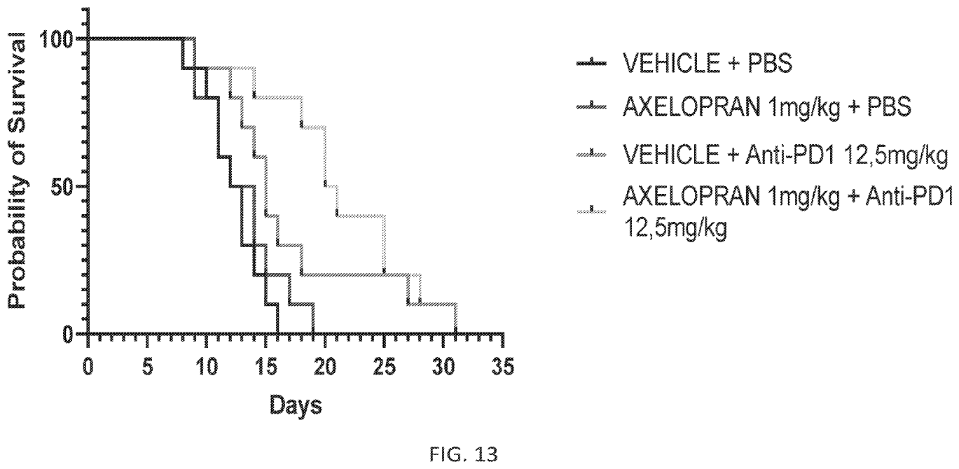

A Kaplan-Meier survival curve of mice injected with human colon cancer MC-38 cells and treated with axelopran (1 mg/kg), anti-PD-1 (2.5 mg/kg) or both agents together is shown in FIG. 13.

Both axelopran and anti-PD-1 individually caused numerically (10-16%) (though not statistically significant) slower tumor progression while the combination of both agents together significantly reduced tumor size by 53% at Day 13 of treatment. These effects were reflected in the mean growth rates over the initial 8 days of treatments (Table 2-3). Associated with the reduction in tumor progression was a 64% increase in survival from 12.5 days in the vehicle-treated control animals to 20.5 days in animals treated with axelopran plus anti-PD-1.

TABLE 2-3
Summary of tumor progression and survival metrics of mice injected
with colon cancer MC-38 cells in the right flank and treated
with axelopran, anti-PD-1 or axelopran plus anti-PD-1.

| | | Vehicle | | | Axelopran + |
|---|---|---|---|---|---|
| | | Control | Axelopran | anti-PD-1 | anti-PD-1 |
| Tumor Size | Mean | 1,626 | 1,479 | 1,376 | 766 |
| @ day 13 | St Dev | 494 | 561 | 529 | 507 |
| (mm³) | | | | | |
| | P-value vs | | n.s | n.s | 0.001 |
| | control | | | | |
| Growth Rate | Mean | 92.5 | 84.5 | 77.7 | 46.3 |
| (mm³/day) | St Dev | 46.5 | 47.7 | 34.7 | 44.4 |
| | P-value vs | | n.s. | n.s. | 0.035 |
| | control | | | | |
| Survival | Median | 12.5 | 13.0 | 15.0 | 20.5 |
| (days) | P-value vs | | n.s. | n.s. | 0.0018 |
| | control | | | | |

The testing in this Example confirms that the MOR antagonist axelopran inhibits tumor growth in the absence of exogenous opioids in at least a melanoma or breast cancer. The testing further establishes that axelopran inhibits metastasis in the absence of exogenous opioids in at least a melanoma or breast cancer. Furthermore, the testing establishes that in the absence of a microbiome, the MOR antagonist activity of axelopran is additive with anti-PD-1 treatment. In addition, the testing establishes that in the presence of a microbiome, the MOR antagonist activity of axelopran activity is synergistic with anti-PD-1 treatment in at least colon cancer.

### Syngeneic Mouse Plus Pancreatic Cancer

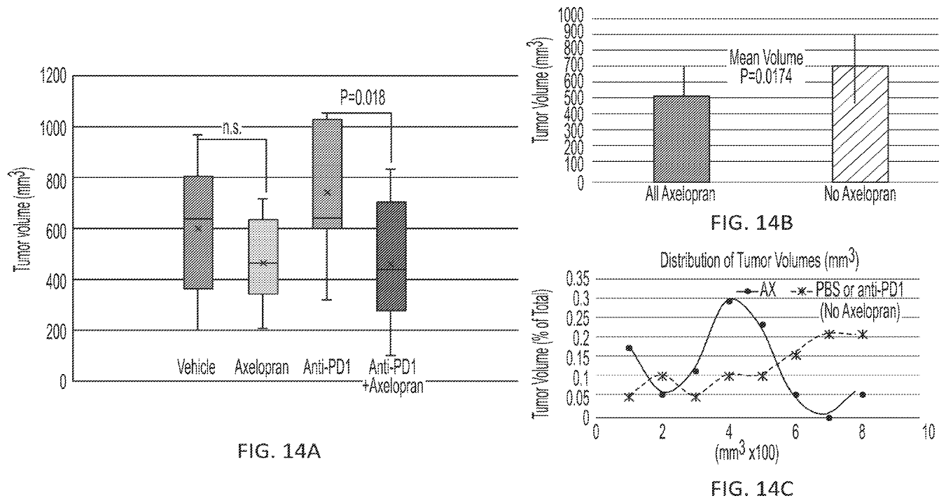

Mouse pancreatic tumor cells (PAN02 cells), maintained in RPMI 1640 media supplemented with 10% FBS and 2 mmol/L L-glutamine, were amplified in vitro prior to implantation. On the day of injection, cells were harvested, counted including a trypan blue viability dye (cut-off 80%), and resuspended in PBS at the appropriate concentration. The cells (5×10⁶ in 200 μL PBS) were injected subcutaneously in the right flank of Female mice (C57BL6), 5 weeks of age (18-22 gm body weight) within 30 minutes after harvesting. After 9 days of tumor growth, mice were randomized into treatment groups at 10 animals/group. Treatments included vehicle (PBS), axelopran given as 1 mg/kg, p.o. daily, mouse anti-PD-1 given as 12.5 mg/kg i.p., 3x/week and axelopran plus mouse anti-PD-1. Animals were weighed thrice weekly and tumor growth measured using callipers as described previously for the colon cancer experiments. The results are shown in FIGS. 14A-14C. As measured at day 23, inhibition of tumor progression was most prominent in axelopran-treated mice (FIGS. 14A,B) and significantly so when axelopran was used in the presence of anti-PD-1. As shown in FIG. 14C the distribution of tumor volumes was lower and more uniform in animals treated with axelopran compared to animals not treated with axelopran.

### ILLUSTRATIVE EMBODIMENTS

Provided here are illustrative embodiments of the disclosed technology. These embodiments are illustrative only and do not limit the scope of the present disclosure or of the claims attached.

### Embodiment 1

A method of reducing or inhibiting endothelial cell growth comprising contacting an endothelial cell with a Mu opioid receptor (MOR) antagonist.

### Embodiment 2

The method of embodiment 1, wherein the method inhibits endothelial cell growth.

### Embodiment 3

A method of reducing angiogenesis in a patient in need thereof comprising administering a MOR antagonist to the patient.

### Embodiment 4

The method of embodiment 3, wherein the method inhibits angiogenesis.

### Embodiment 5

The method of embodiments 3 or 4, wherein the method reduces or inhibits ocular angiogenesis.

### Embodiment 6

The method of embodiments 3 or 4, wherein the patient has psoriasis.

### Embodiment 7

A method of reducing angiogenesis in a cancer patient comprising administering a MOR antagonist to the cancer patient.

### Embodiment 8

The method of embodiment 7, wherein the method inhibits angiogenesis.

### Embodiment 9

A method of reducing tumor growth in a cancer patient comprising administering a MOR antagonist to the cancer patient.

### Embodiment 10

The method of embodiment 9, wherein the method inhibits tumor growth.

### Embodiment 11

A method of reducing metastasis in a cancer patient comprising administering a MOR antagonist to the cancer patient.

### Embodiment 12

The method of embodiment 11, wherein the method inhibits metastasis.

### Embodiment 13

A method of improving the immune response to a tumor comprising administering a MOR antagonist to a cancer patient.

### Embodiment 14

The method of embodiment 13, wherein the method increases infiltration of immune cells into the tumor.

### Embodiment 15

The method of embodiment 13, wherein the method increases infiltration of NK cells, lymphocytes and/or monocytes/macrophages.

### Embodiment 16

The method of embodiment 13, wherein the method increases infiltration of CD3⁺, CD244⁺, and MMD⁺ immune cells.

### Embodiment 17

The method of any one of embodiments 1-16, wherein the cancer is a melanoma, colon, or breast cancer.

### Embodiment 18

The method of embodiments any one of embodiments 1-17, wherein the patient is not receiving opioid treatment.

### Embodiment 19

The method of any one of embodiments 1-18, wherein the method does not comprise administering an opioid.

### Embodiment 20

The method of any one of embodiments 1-19, wherein the MOR antagonist does not comprise methylnaltrexone.

### Embodiment 21

The method of any one of embodiments 1-20, wherein the MOR antagonist is axelopran, naloxegol, naldemedine, alvimopan, or combinations thereof.

### Embodiment 22

The method of embodiment 21, wherein the MOR antagonist is axelopran.

### Embodiment 23

The method of any one of embodiments 1-23, wherein the method comprises orally administering the MOR antagonist.

### Embodiment 24

The method of any one of embodiments 1-23, wherein the method comprises subcutaneously administering the MOR antagonist.

### Embodiment 25

The method of any one of embodiments 1-23, wherein the method comprises intravitreally administering the MOR antagonist.

### Embodiment 26

The method of any one of embodiments 1-23, wherein the method comprises to topically administering the MOR antagonist.

### Embodiment 27

The method of any one of embodiments 1-23, wherein the method comprises administering a pharmaceutical composition comprising the MOR antagonist and a pharmaceutically acceptable carrier.

### Embodiment 28

The method of embodiment 19, wherein the pharmaceutical composition is formulated for modified release.

### Embodiment 29

A method of treating cancer comprising administering axelopran, naloxegol, or combinations thereof to a cancer patient.

### Embodiment 30

The method of embodiment 29, wherein the method comprises administering axelopran.

### Embodiment 31

The method of embodiments 29 or 30, wherein the method reduces angiogenesis, tumor growth, and/or metastasis.

### Embodiment 32

The method of embodiment 32, wherein the method inhibits angiogenesis, tumor growth, and/or metastasis.

### Embodiment 33

The method of any one of embodiments 29-32, wherein the method does not comprise administering an exogenous opioid.

### Embodiment 34

The method of any one of embodiments 29-32, wherein the patient is not receiving opioid treatment.

### Embodiment 35

The method of any one of embodiments 29-32, wherein cancer is a melanoma, colon, pancreatic, or breast cancer.

### Embodiment 36

The method of any one of embodiments 29-35, wherein the method comprises orally administering the MOR antagonist.

### Embodiment 37

The method of any one of embodiments 29-35, wherein the method comprises subcutaneously administering the MOR antagonist.

### Embodiment 38

The method of any one of embodiments 29-35, wherein the method comprises administering a pharmaceutical composition comprising the MOR antagonist and a pharmaceutically acceptable carrier.

### Embodiment 39

The method of embodiment 38, wherein the pharmaceutical composition is formulated for modified release, topical administration, or intravitreal injection.

### Embodiment 40

The method of any one of embodiments 7-39, wherein the cancer is refractive to treatment with a checkpoint inhibitor.

The methods of any one of embodiments 1-40 can also include administering a checkpoint inhibitor and/or an inhibitor of angiogenesis, such as e.g. an inhibitor of VEGF.

### Embodiment 41

A method of reducing tumor size comprising contacting a tumor with axelopran, naloxegol, or combinations thereof.

### Embodiment 42

A method of treating cancer comprising administering a MOR antagonist and a checkpoint inhibitor and/or an inhibitor of VEGF to a cancer patient.

### Embodiment 43

The method of embodiment 42, wherein the MOR antagonist and the checkpoint inhibitor and/or an inhibitor of VEGF synergistically treat the cancer.

### Embodiment 44

A method of improving the immune response to a tumor comprising administering a MOR antagonist and a checkpoint inhibitor and/or an inhibitor of VEGF to a cancer patient.

### Embodiment 45

The method of embodiment 44, wherein the method increases infiltration of immune cells into the tumor.

### Embodiment 46

The method of embodiment 45, wherein the method increases infiltration of NK cells, lymphocytes and/or monocytes/macrophages.

### Embodiment 47

The method of embodiment 45, wherein the method increases infiltration of CD3⁺, CD244⁺, and MMD⁺ immune cells.

### Embodiment 48

The method of embodiment 45, wherein the MOR antagonist and the checkpoint inhibitor synergistically improve the immune response to the tumor.

### Embodiment 49

The method of any one of embodiments 42-49, wherein the MOR antagonist comprises axelopran, naloxegol, methylnaltrexone, or combinations thereof.

### Embodiment 50

The method of embodiment 49, the MOR antagonist is axelopran.

### Embodiment 51

The method of any one of embodiments 42-50, wherein the checkpoint inhibitor is an inhibitor of the PD-1/PD-L1 pathway.

### Embodiment 52

The method of embodiment 51, wherein the inhibitor of the PD-1/PD-L1 pathway is an antibody.

### Embodiment 53

The method of embodiment 52, wherein the antibody is an antibody that binds to PD-1.

### Embodiment 54

The method of embodiment 53, wherein the antibody is a humanized antibody.

### Embodiment 55

The method of embodiment 53, wherein the human antibody is pembrolizumab.

### Embodiment 56

The method of any one of embodiments 42-55, wherein the method comprises orally or subcutaneously administering the MOR antagonist.

### Embodiment 57

The method of any one of embodiments 42-56, wherein the method comprises administering a pharmaceutical composition comprising the MOR antagonist and a pharmaceutically acceptable carrier.

### Embodiment 58

The method of embodiment 57, wherein the pharmaceutical composition is formulated for modified release.

### Embodiment 59

The method of any one of embodiments 42-58, wherein the checkpoint inhibitor is administered concurrently, before, or after the MOR antagonist.

### Embodiment 60

The method of any one of embodiments 42-59, wherein the method comprises administering a checkpoint inhibitor.

### Embodiment 61

The method of any one of embodiments 42-60, wherein the method comprises administering an inhibitor of VEGF.

### Embodiment 62

The method of any one of embodiments 42-60, wherein the inhibitor of VEGF is an anti-VEGF antibody.

### Embodiment 63

The method of embodiment 62, wherein the antibody is humanized.

### Embodiment 64

The method of embodiments 62 or 63, wherein the antibody is an anti-VEGF-A antibody.

### Embodiment 65

The method any one of embodiments 61-64, wherein the inhibitor of VEGF is bevacizumab.

### Embodiment 66

The method of any one of embodiments 61-65, comprising intravenously injecting the inhibitor of VEGF.

### Embodiment 67

The method of any one of embodiments 61-66, wherein the inhibitor of VEGF is administered concurrently, before, or after the MOR antagonist.

### Embodiment 68

The method of any one of embodiments 61-67, wherein the inhibitor of VEGF is administered concurrently, before, or after the checkpoint inhibitor.

### Embodiment 69

A kit comprising a MOR antagonist and a checkpoint inhibitor and/or an inhibitor of VEGF.

### Embodiment 70

The kit of embodiment 69, wherein the checkpoint inhibitor is an inhibitor of the PD-1/PD-L1 pathway.

### Embodiment 71

The kit of embodiment 69, wherein the kit comprises a pharmaceutical a pharmaceutical composition comprising the MOR antagonist and a pharmaceutically acceptable carrier.

### Embodiment 72

The kit of embodiment 71, wherein the pharmaceutical composition further comprises an inhibitor of VEGF.

### Embodiment 73

The kit of embodiments 71 or 72, wherein the pharmaceutical composition is formulated for modified release, topical administration, or intravitreal injection.

### Embodiment 74

A composition comprising a synergistic amount of a MOR antagonist and an inhibitor of the PD-1/PD-L1 pathway.

### Embodiment 75

The composition of embodiment 74, wherein the MOR antagonist comprises axelopran, naloxegol, methylnaltrexone, or combinations thereof.

### Embodiment 76

The composition of embodiment 74, the MOR antagonist is axelopran.

### Embodiment 77

The composition of any one of embodiments 74-76, wherein the inhibitor of the PD-1/PD-L1 pathway is an antibody.

### Embodiment 78

The composition of any one of embodiments 74-77, wherein the antibody is an antibody that binds to PD-1.

### Embodiment 79

The composition of embodiment 78, wherein the antibody is a humanized antibody.

### Embodiment 80

The method of embodiment 79, wherein the human antibody is pembrolizumab.

### Embodiment 81

Use of axelopran, naloxegol, or combinations thereof in the manufacture of medicament for treating cancer.

### Embodiment 82

Use of axelopran, naloxegol, or combinations thereof for treating cancer.

### Embodiment 83

Use of a MOR antagonist and an inhibitor of the PD-1/PD-L1 pathway in the manufacture of a medicament for treating cancer.

### Embodiment 84

Use of a MOR antagonist and an inhibitor of the PD-1/PD-L1 pathway for treating cancer.

### Embodiment 85

Use of a MOR antagonist and an inhibitor of VEGF in the manufacture of a medicament for treating cancer.

### Embodiment 86

Use of a MOR antagonist and an inhibitor of VEGF for treating cancer.

### Embodiment 87

Use of a MOR antagonist, an inhibitor of the PD-1/PD-L1 pathway, and an inhibitor of VEGF in the manufacture of a medicament for treating cancer.

### Embodiment 88

Use of a MOR antagonist, an inhibitor of the PD-1/PD-L1 pathway, and an inhibitor of VEGF for treating cancer.

### Embodiment 89

The use of any one of embodiments 85-88, wherein the VEGF inhibitor is anti-VEGF-A antibody.

### Embodiment 90

The use of embodiment 89, wherein the inhibitor of VEGF is bevacizumab.

### Embodiment 91

The use of any one of embodiments 83-90, wherein the inhibitor of the PD-1/PD-L1 pathway is an antibody.

### Embodiment 92

The use of any one of embodiments 83-91, wherein the antibody is an antibody that binds to PD-1.

### Embodiment 93

The use of embodiment 92, wherein the antibody is a humanized antibody.

### Embodiment 94

The use of embodiment 93, wherein the human antibody is pembrolizumab.

### Embodiment 95

Use of a MOR antagonist for treating cancer in a patient undergoing therapy inhibiting the PD-1/PD-L1 pathway.

### Embodiment 96

The use of any one of embodiments 83-95, wherein the MOR antagonist comprises axelopran, naloxegol, methylnaltrexone, or combinations thereof.

### Embodiment 97

The use of embodiment 96, wherein the MOR antagonist is axelopran.

### Embodiment 98

A combination comprising a MOR antagonist and an inhibitor of the PD-1/PD-L1 pathway for use in a method of treating cancer, the method comprising administering the combination to a subject in need thereof.

### Embodiment 99

The combination of embodiment 98, wherein the MOR antagonist is axelopran.

### Embodiment 100

The combination of embodiments 98 or 99, wherein the inhibitor of the PD-1/PD-L1 pathway is pembrolizumab.

### Embodiment 101

The combination of embodiments 98 or 99, wherein the combination further comprises an inhibitor of VEGF.

The combination of any one of embodiments 98-101, wherein the inhibitor of VEGF is bevacizumab. The disclosures of each patent, patent application, and publication cited or described herein are hereby incorporated herein by reference, each in its entirety, for all purposes.

What is claimed:

1. A method of reducing or inhibiting endothelial cell growth comprising contacting an endothelial cell with a Mu opioid receptor (MOR) antagonist.

2. The method of claim 1, wherein the method inhibits endothelial cell growth.

3. A method of reducing angiogenesis in a patient in need thereof comprising administering a MOR antagonist to the patient.

4. The method of claim 3, wherein the method inhibits angiogenesis.

5. The method of claim 3 or 4, wherein the method reduces or inhibits ocular angiogenesis.

6. The method of claim 3 or 4, wherein the patient has psoriasis.

7. A method of reducing angiogenesis in a cancer patient comprising administering a MOR antagonist to the cancer patient.

8. The method of claim 7, wherein the method inhibits angiogenesis.

9. A method of reducing tumor growth in a cancer patient comprising administering a MOR antagonist to the cancer patient.

10. The method of claim 9, wherein the method inhibits tumor growth.

11. A method of reducing metastasis in a cancer patient comprising administering a MOR antagonist to the cancer patient.

12. The method of claim 11, wherein the method inhibits metastasis.

13. A method of improving the immune response to a tumor comprising administering a MOR antagonist to a cancer patient.

14. The method of claim 13, wherein the method increases infiltration of immune cells into the tumor.

15. The method of claim 13, wherein the method increases infiltration of NK cells, lymphocytes and/or monocytes/macrophages.

16. The method of claim 13, wherein the method increases infiltration of CD3⁺, CD244⁺, and MMD⁺ immune cells.

17. The method of any one of claims 1-16, wherein the cancer is a melanoma, colon, pancreatic, or breast cancer.

18. The method of claims any one of claims 1-17, wherein the patient is not receiving opioid treatment.

19. The method of any one of claims 1-18, wherein the method does not comprise administering an opioid.

20. The method of any one of claims 1-19, wherein the MOR antagonist does not comprise methylnaltrexone.

21. The method of any one of claims 1-20, wherein the MOR antagonist is axelopran, naloxegol, naldemedine, alvimopan, or combinations thereof.

22. The method of claim 21, wherein the MOR antagonist is axelopran.

23. The method of any one of claims 1-23, wherein the method comprises orally administering the MOR antagonist.

24. The method of any one of claims 1-23, wherein the method comprises subcutaneously administering the MOR antagonist.

25. The method of any one of claims 1-23, wherein the method comprises intravitreally administering the MOR antagonist.

26. The method of any one of claims 1-23, wherein the method comprises to topically administering the MOR antagonist.

27. The method of any one of claims 1-23, wherein the method comprises administering a pharmaceutical composition comprising the MOR antagonist and a pharmaceutically acceptable carrier.

28. The method of claim 19, wherein the pharmaceutical composition is formulated for modified release.

29. A method of treating cancer comprising administering axelopran, naloxegol, or combinations thereof to a cancer patient.

30. The method of claim 29, wherein the method comprises administering axelopran.

31. The method of claim 29 or 30, wherein the method reduces angiogenesis, tumor growth, and/or metastasis.

32. The method of claim 32, wherein the method inhibits angiogenesis, tumor growth, and/or metastasis.

33. The method of any one of claims 29-32, wherein the method does not comprise administering an exogenous opioid.

34. The method of any one of claims 29-32, wherein the patient is not receiving opioid treatment.

35. The method of any one of claims 29-32, wherein cancer is a melanoma, colon, pancreatic, or breast cancer.

36. The method of any one of claims 29-35, wherein the method comprises orally administering the MOR antagonist.

37. The method of any one of claims 29-35, wherein the method comprises subcutaneously administering the MOR antagonist.

38. The method of any one of claims 29-35, wherein the method comprises administering a pharmaceutical composition comprising the MOR antagonist and a pharmaceutically acceptable carrier.

39. The method of claim 38, wherein the pharmaceutical composition is formulated for modified release, topical administration, or intravitreal injection.

40. The method of any one of claims 7-39, wherein the cancer is refractive to treatment with a checkpoint inhibitor.

41. A method of reducing tumor size comprising contacting a tumor with axelopran, naloxegol, or combinations thereof.

42. A method of treating cancer comprising administering a MOR antagonist and a checkpoint inhibitor and/or an inhibitor of VEGF to a cancer patient.

43. The method of claim 42, wherein the MOR antagonist and the checkpoint inhibitor and/or an inhibitor of VEGF synergistically treat the cancer.

44. A method of improving the immune response to a tumor comprising administering a MOR antagonist and a checkpoint inhibitor and/or an inhibitor of VEGF to a cancer patient.

45. The method of claim 44, wherein the method increases infiltration of immune cells into the tumor.

46. The method of claim 45, wherein the method increases infiltration of NK cells, lymphocytes and/or monocytes/macrophages.

47. The method of claim 45, wherein the method increases infiltration of CD3⁺, CD244⁺, and MMD⁺ immune cells.

48. The method of claim 45, wherein the MOR antagonist and the checkpoint inhibitor synergistically improve the immune response to the tumor.

49. The method of any one of claims 42-49, wherein the MOR antagonist comprises axelopran, naloxegol, methylnaltrexone, or combinations thereof.

50. The method of claim 49, the MOR antagonist is axelopran.

51. The method of any one of claims 42-50, wherein the checkpoint inhibitor is an inhibitor of the PD-1/PD-L1 pathway.

52. The method of claim 51, wherein the inhibitor of the PD-1/PD-L1 pathway is an antibody.

53. The method of claim 52, wherein the antibody is an antibody that binds to PD-1.

54. The method of claim 53, wherein the antibody is a humanized antibody.

55. The method of claim 53, wherein the human antibody is pembrolizumab.

56. The method of any one of claims 42-55, wherein the method comprises orally or subcutaneously administering the MOR antagonist.

57. The method of any one of claims 42-56, wherein the method comprises administering a pharmaceutical composition comprising the MOR antagonist and a pharmaceutically acceptable carrier.

58. The method of claim 57, wherein the pharmaceutical composition is formulated for modified release.

59. The method of any one of claims 42-58, wherein the checkpoint inhibitor is administered concurrently, before, or after the MOR antagonist.

60. The method of any one of claims 42-59, wherein the method comprises administering a checkpoint inhibitor.

61. The method of any one of claims 42-60, wherein the method comprises administering an inhibitor of VEGF.

62. The method of any one of claims 42-60, wherein the inhibitor of VEGF is an anti- VEGF antibody.

63. The method of claim 62, wherein the antibody is humanized.

64. The method of claim 62 or 63, wherein the antibody is an anti-VEGF-A antibody.

65. The method any one of claims 61-64, wherein the inhibitor of VEGF is bevacizumab.

66. The method of any one of claims 61-65, comprising intravenously injecting the inhibitor of VEGF.

67. The method of any one of claims 61-66, wherein the inhibitor of VEGF is administered concurrently, before, or after the MOR antagonist.

68. The method of any one of claims 61-67, wherein the inhibitor of VEGF is administered concurrently, before, or after the checkpoint inhibitor.

69. A kit comprising a MOR antagonist and a checkpoint inhibitor and/or an inhibitor of VEGF.

70. The kit of claim 69, wherein the checkpoint inhibitor is an inhibitor of the PD-1/PD-1 pathway.

71. The kit of claim 69, wherein the kit comprises a pharmaceutical a pharmaceutical composition comprising the MOR antagonist and a pharmaceutically acceptable carrier.

72. The kit of claim 71, wherein the pharmaceutical composition further comprises an inhibitor of VEGF.

73. The kit of claim 71 or 72, wherein the pharmaceutical composition is formulated for modified release, topical administration, or intravitreal injection.

74. A composition comprising a synergistic amount of a MOR antagonist and an inhibitor of the PD-1/PD-L1 pathway.

75. The composition of claim 74, wherein the MOR antagonist comprises axelopran, naloxegol, methylnaltrexone, or combinations thereof.

76. The composition of claim 74, the MOR antagonist is axelopran.

77. The composition of any one of claims 74-76, wherein the inhibitor of the PD-1/PD-L1 pathway is an antibody.

78. The composition of any one of claims 74-77, wherein the antibody is an antibody that binds to PD-1.

79. The composition of claim 78, wherein the antibody is a humanized antibody.

80. The method of claim 79, wherein the human antibody is pembrolizumab.

81. Use of axelopran, naloxegol, or combinations thereof in the manufacture of medicament for treating cancer.

82. Use of axelopran, naloxegol, or combinations thereof for treating cancer.

83. Use of a MOR antagonist and an inhibitor of the PD-1/PD-L1 pathway in the manufacture of a medicament for treating cancer.

84. Use of a MOR antagonist and an inhibitor of the PD-1/PD-L1 pathway for treating cancer.

85. Use of a MOR antagonist and an inhibitor of VEGF in the manufacture of a medicament for treating cancer.

86. Use of a MOR antagonist and an inhibitor of VEGF for treating cancer.

87. Use of a MOR antagonist, an inhibitor of the PD-1/PD-L1 pathway, and an inhibitor of VEGF in the manufacture of a medicament for treating cancer.

88. Use of a MOR antagonist, an inhibitor of the PD-1/PD-L1 pathway, and an inhibitor of VEGF for treating cancer.

89. The use of any one of claims 85-88, wherein the VEGF inhibitor is anti-VEGF-A antibody.

90. The use of claim 89, wherein the inhibitor of VEGF is bevacizumab.

91. The use of any one of claims 83-90, wherein the inhibitor of the PD-1/PD-L1 pathway is an antibody.

92. The use of any one of claims 83-91, wherein the antibody is an antibody that binds to PD-1.

93. The use of claim 92, wherein the antibody is a humanized antibody. 94 The use of claim 93, wherein the human antibody is pembrolizumab.

95. Use of a MOR antagonist for treating cancer in a patient undergoing therapy inhibiting the PD-1/PD-L1 pathway.

96. The use of any one of claims 83-95, wherein the MOR antagonist comprises axelopran, naloxegol, methylnaltrexone, or combinations thereof.

97. The use of claim 96, wherein the MOR antagonist is axelopran.

98. A combination comprising a MOR antagonist and an inhibitor of the PD-1/PD-L1 pathway for use in a method of treating cancer, the method comprising administering the combination to a subject in need thereof.

99. The combination of claim 98, wherein the MOR antagonist is axelopran.

100. The combination of claim 98 or 99, wherein the inhibitor of the PD-1/PD-L1 pathway is pembrolizumab.

101. The combination of claim 98 or 99, wherein the combination further comprises an inhibitor of VEGF.

102. The combination of any one of claims 98-101, wherein the inhibitor of VEGF is bevacizumab.
